(19) United States
(12) Patent Application Publication
(10) Pub. No.: US 2026/0105486 A1
(43) Pub. Date: Apr. 16, 2026

(54) Automotive AI Sensor Fusion

(71) Applicant: Panasonic Automotive System America, LLC., Peachtree City, GA (US)

(72) Inventors: ERIC SHUFRO, Peachtree City, GA (US); JAMES LOCK, PEACHTREE CITY, GA (US)

(21) Appl. No.: 19/356,778

(22) Filed: Oct. 13, 2025

Related U.S. Application Data
(60) Provisional application No. 63/707,255, filed on Oct. 15, 2024.

(51) Int. Cl. G06Q 30/0242; A61B 5/00; A61B 5/16; A61B 5/18; B60Q 9/00; G06V 10/774; G06V 20/59; G10L 25/63

(52) U.S. Cl. CPC G06Q 30/0242; A61B 5/0002; A61B 5/165; A61B 5/18; A61B 5/6893; B60Q 9/00; G06V 10/774; G06V 20/597; G10L 25/63; A61B 2503/22; A61B 2562/0204

(57) ABSTRACT

A motor vehicle includes a plurality of sensors that collect data indicative of an emotional state of an occupant of the motor vehicle. The sensors transmit sensor signals including the data. An infotainment system plays advertisements such that the occupant can hear the advertisements. An electronic processor is communicatively coupled to the sensing devices and to the infotainment system. The electronic processor receives the sensor signals, and wirelessly transmits a report signal indicative of the received sensor signals and indicative of which of the advertisements correspond to the received sensor signals. The report signal is wirelessly transmitted to a remote datacenter which runs an artificial intelligence-based algorithm to determine emotional states of the occupant of the motor vehicle as a result of the advertisements. The running of the algorithm is based on the received sensor signals.

### CROSS-REFERENCE TO RELATED APPLICATIONS

This application claims benefit of U.S. Provisional Application No. 63/707,255, filed on Oct. 15, 2024, the disclosure of which is hereby incorporated by reference in its entirety for all purposes.

### BACKGROUND OF THE INVENTION

### 1. Field of the Invention

The present invention relates to a driving monitoring system in a motor vehicle.

### 2. Description of the Related Art

Advertisers lack insight into the effectiveness of their radio advertisements in capturing and maintaining the attention of drivers. Traditionally, advertisers rely on indirect metrics like audience reach and listener demographics to gauge the impact of their ads. However, these metrics don't provide direct feedback on whether the advertisement is resonating with the audience emotionally.

### SUMMARY OF THE INVENTION

The invention may include an artificial intelligence (AI)-based (e.g., machine learning-based) driving monitoring system adapted to feed driver state information to a connected radio platform. The driving monitoring system may utilize facial recognition and emotion detection technology. The inventive system may include cameras installed within the vehicle that continuously monitor the driver's facial expressions and gestures to determine their emotional state, such as happiness, sadness, or annoyance. The invention may utilize other vehicle sensors to feed an AI data model and increase its accuracy. The other vehicle sensors may include sensors to produce vehicle data or engine data, detect location, speed or G-force, microphones to perform voice control, cameras to perform face and body vision and eye tracking, controls to determine a vehicle interface state and perform monitoring, seat or occupancy sensors, steering wheel sensors, and controls to receive infotainment on-screen touch input and/or physical button interaction.

The collected data on the driver's emotional state may then be processed by AI algorithms to categorize the data into different emotional states. This information may be transmitted to a connected radio platform, which would analyze the driver's emotional state in relation to the advertisements being played on the radio. For example, if the system detects that the driver is happy while a specific advertisement is playing, it may be inferred that the advertisement has made a positive impact on the driver. Conversely, if the driver appears annoyed or disinterested during an advertisement, it may indicate that the advertisement is not effectively resonating with the driver. This feedback could then be utilized by advertisers to gauge the effectiveness of their advertisements and tailor their marketing strategies accordingly.

There are other AI use cases of the invention in addition to advertisement reaction and feedback, such as smart personalization, security, health monitoring to detect illness, mood, heart rate, intoxication, etc., and user interface (UI) optimization and ease of use.

This invention differs from other products or methods used for similar purposes by offering a more direct and real-time feedback mechanism for advertisers to assess the effectiveness of their radio advertisements. While traditional methods rely on indirect metrics like listener demographics and reach, this invention leverages AI-driven driving monitoring systems to analyze the emotional states of drivers in response to specific advertisements.

Compared to existing methods such as surveys or focus groups, which may suffer from biases or inaccuracies due to self-reporting or small sample sizes, this invention provides objective data on driver reactions by monitoring their facial expressions and gestures. This real-time feedback allows advertisers to make timely adjustments to their advertising strategies to better resonate with their target audience.

Furthermore, on-screen interactions prompting the user to indicate whether they liked or disliked an advertisement could be used to train local and cloud-based machine learning algorithms.

The car is a fantastic environment for driver monitoring (having similar access to the user that an Apple Vision Pro has). AI can be better trained and hence make better conclusions by taking advantage of all this information available in the car, such as video images of the driver and other sensor data that is uniquely available in a constrained space.

This invention also has the benefit of scalability and efficiency. By automating the process of collecting and analyzing driver state information, advertisers can gather insights from a larger and more diverse pool of drivers without the need for manual intervention or additional resources. Overall, the invention provides advertisers with a more accurate, timely, and scalable method for evaluating the impact of their radio advertisements, leading to improved decision-making and ultimately, more effective advertising campaigns.

The invention may employ audio-based emotion detection. Instead of relying solely on facial recognition technology, the system may utilize audio sensors (e.g., microphones) to analyze the tone and pitch of the driver's voice to infer their emotional state. This provides an additional method for assessing the driver's reaction to radio advertisements. It may also provide an alternative method for assessing the driver's reaction to radio advertisements without the need for visual monitoring.

The invention may utilize wearable devices. Rather than installing cameras within the vehicle, the system may integrate with wearable devices such as smartwatches or headsets equipped with biometric sensors. These devices may continuously monitor physiological indicators like heart rate variability or skin conductance to gauge the driver's emotional response to advertisements.

The invention may also utilize natural language processing. In addition to analyzing facial expressions or voice tone, the system may incorporate natural language processing (NLP) algorithms to analyze the content and sentiment of any verbal responses or conversations within the vehicle. This may provide further insights into the driver's emotional state and engagement with the advertisements.

The invention may perform cloud-based processing. As an alternative to processing the data locally within the vehicle, the system may transmit the raw sensor data to a cloud-based platform for analysis. Cloud-based processing may enable more sophisticated AI algorithms to be applied, as well as facilitate centralized data storage and processing for scalability and accessibility.

The invention may implement multi-modal fusion of sensor outputs. Combining multiple sensor modalities such as facial recognition, audio analysis, and physiological monitoring may enhance the accuracy and reliability of the system's assessment of the driver's emotional state. By fusing data from different sources, the system may provide a more comprehensive report of the driver's response to radio advertisements.

In one embodiment, the inventive system utilizes an in-vehicle AI-based voice assistant to ask the user, “Did you like this ad?” The direct feedback may be collected and mapped against the perceived facial recognition for further training of the driver monitoring system and its ability to read emotions linked to advertisements.

The invention comprises, in one form thereof, a motor vehicle including a plurality of sensors that collect data indicative of an emotional state of an occupant of the motor vehicle. The sensors transmit sensor signals including the data. An infotainment system plays advertisements such that the occupant can hear the advertisements. An electronic processor is communicatively coupled to the sensing devices and to the infotainment system. The electronic processor receives the sensor signals, and wirelessly transmits a report signal indicative of the received sensor signals and indicative of which of the advertisements correspond to the received sensor signals. The report signal is wirelessly transmitted to a remote datacenter which runs an artificial intelligence-based algorithm to determine emotional states of the occupant of the motor vehicle as a result of the advertisements. The running of the algorithm is based on the received sensor signals.

The invention comprises, in another form thereof, a method of evaluating advertisements in a motor vehicle, including playing the advertisements such that an occupant of the motor vehicle can hear the advertisements. Sensor data indicative of an emotional state of the occupant of the motor vehicle is collected. A report signal is wirelessly transmitted to a remote datacenter. The report signal is indicative of the sensor data and indicative of which of the advertisements correspond to the sensor data. An artificial intelligence-based algorithm is run to determine emotional states of the occupant of the motor vehicle as a result of the advertisements. The algorithm is run based on the report signal. The algorithm is run at a location remote from the motor vehicle.

The invention comprises, in yet another form thereof, an advertisement evaluation arrangement for an automotive environment. The arrangement includes a motor vehicle having a plurality of sensors that collect data indicative of emotional states of an occupant of the motor vehicle. The sensors transmit sensor signals including the data. An infotainment system plays advertisements such that the occupant can hear the advertisements. An electronic processor is communicatively coupled to the sensing devices and to the infotainment system. The electronic processor receives the sensor signals and wirelessly transmits a report signal indicative of the received sensor signals and indicative of which of the advertisements correspond to the received sensor signals. A datacenter is disposed remote from the motor vehicle and receives the report signal. The datacenter runs an artificial intelligence-based algorithm to determine the emotional states of the occupant of the motor vehicle as a result of the advertisements. The algorithm is run based on the received report signal.

### BRIEF DESCRIPTION OF THE DRAWINGS

The above-mentioned and other features and objects of this invention, and the manner of attaining them, will become more apparent and the invention itself will be better understood by reference to the following description of embodiments of the invention taken in conjunction with the accompanying drawings, wherein:

FIG. 1 is a perspective view of the passenger compartment of a motor vehicle including one embodiment of an automotive AI sensor fusion arrangement of the present invention.

FIG. 2 is a block diagram of one embodiment of an automotive AI sensor fusion arrangement of the present invention.

FIG. 3 is a flow chart of one embodiment of a method of the invention for evaluating advertisements in a motor vehicle.

### DETAILED DESCRIPTION

The embodiments hereinafter disclosed are not intended to be exhaustive or limit the invention to the precise forms disclosed in the following description. Rather the embodiments are chosen and described so that others skilled in the art may utilize its teachings.

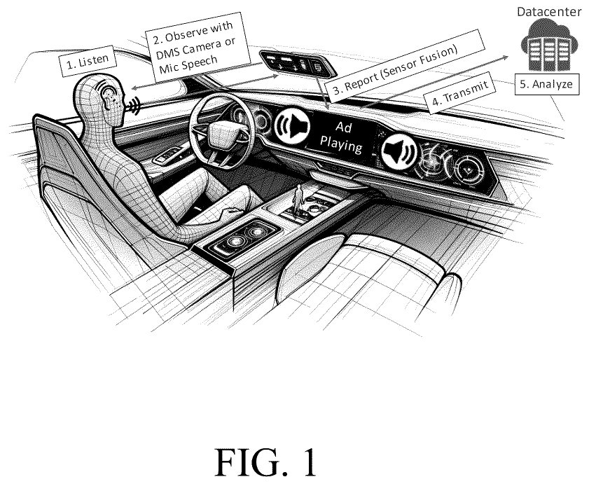

FIG. 1 illustrates a passenger compartment of a motor vehicle including one embodiment of an automotive AI sensor fusion arrangement of the present invention that is used to perform an automotive AI sensor fusion method of the present invention. In a first step of the inventive method, the driver listens to an advertisement or other media content being played on the infotainment system. The content may be played on a radio or online streaming service, for example.

In a second step of the method, the arrangement observes and monitors the driver with cameras or other sensors such as microphones. The observing and monitoring may occur while the advertisement is being played, or within a threshold period of time after the advertisement has ended, such as three seconds, for example.

In a third step of the method, the arrangement combines information (e.g., a fusion of sensor outputs) into a report. The report may include all sensor readings obtained during the advertisement, and within the threshold time period after the advertisement has stopped playing.

In a fourth step of the method, the arrangement transmits the report to the cloud/datacenter for analysis. For example, the report may be transmitted wirelessly through an Internet connection.

In a fifth and final step, the datacenter runs AI models on reported data to identify and correlate the driver's reactions to content. For example, based on the historic data of AI models, the current sensor data can be interpreted to ascertain what emotions the driver is feeling. This behavior analysis can be used in many different ways. In a simplest case, if the driver has a happy reaction, then the advertisement may continue to run on the media. But if the driver is annoyed, then the advertisement may be discontinued and may no longer be run on the media.

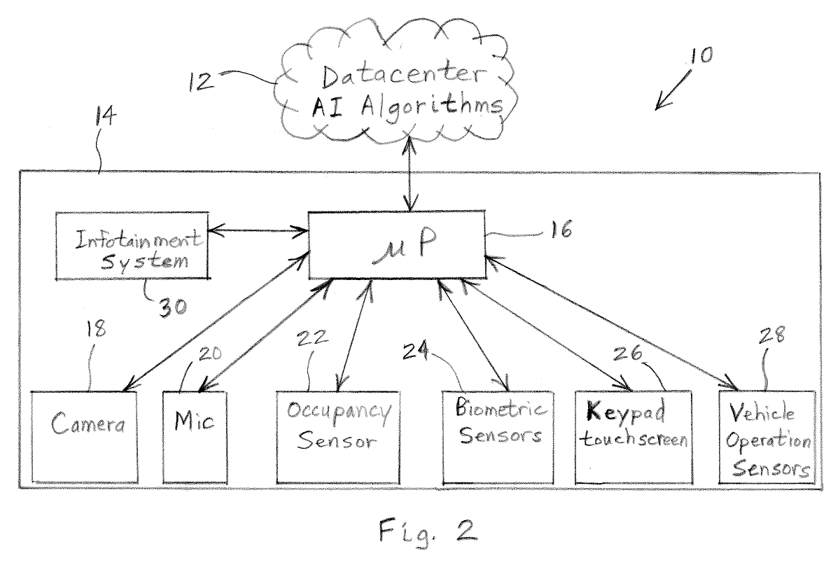

FIG. 2 illustrates one embodiment of an automotive AI sensor fusion arrangement 10 of the present invention, including a cloud-based datacenter 12 and a motor vehicle 14. Datacenter 12 may include servers for storing data and running artificial intelligence algorithms. The artificial intelligence algorithms may be trained on training data from the sensors in vehicle 14, using machine learning, for example, to ascertain the real time emotional state of a driver of vehicle 14 based on real time data from the in-vehicle sensors.

Motor vehicle 14 may include an electronic processor 16 in bi-directional wireless communication with datacenter 12 and in bi-directional communication with a plurality of in-vehicle sensors, such as a camera 18, a microphone 20, an occupancy sensor 22, biometric sensors 24, a tactile user interface 26 such as a keypad and/or touchscreen, and vehicle operation sensors 28. Camera 18 may have a field of view that encompasses the driver's face, arms and hands so that camera 18 can capture images of the driver's facial expressions, hand gestures, eye movements, head turns, eye blinking, mouth movements and lip movements (e.g., for reading the driver's lips to ascertain what he is saying), for example.

Microphone 20 may be positioned to pick up all spoken utterances, gasps, sighs, etc. from the driver. Occupancy sensor 22 may be a force sensor that detects the weight of the driver in the driver's seat. Occupancy sensor 22 may also be a carbon dioxide sensor, a camera, or a touch sensor on the steering wheel, for example.

Biometric sensors 24 may be wearable devices such as Apple watches, bracelets, pendants, etc., that measure the driver's pulse, heart rate, breathing, blood pressure, etc. Tactile user interface 26 may be on the center console of the vehicle or any other location that is within arm's reach of the driver.

Vehicle operation sensors 28 may include speedometers, acceleration sensors, deacceleration sensors, brake sensors, steering sensors, location sensors (e.g., GPS) and user-actuatable infotainment system controls. For example, the driver turning up the media playback volume during an advertisement may indicate that the driver likes the advertisement. Conversely, the driver turning down the media playback volume during an advertisement, turning off the advertisement, or switching to different media content may indicate that the driver dislikes the advertisement.

Motor vehicle 14 may include an infotainment system 30 that may play advertisements from radio broadcasts or streaming services. The time periods in which advertisements are played may be matched or correlated to corresponding sensor signals that are transmitted during the advertisements or in a short threshold time period after the advertisement ends, such as three seconds. Thus, the sensor signals may most likely reflect emotions that the driver presents due to the playing of the corresponding advertisement. Each advertisement may include time stamps that are matched with corresponding time stamps of the sensor signals. Alternatively, processor 16 may match sensor signals to advertisements that are playing at the times that the sensor signals are received. Processor 16 may keep a record of which sensor signals correspond to which advertisements.

During use, after receiving the sensor data signals from the various in-vehicle sensors, processor 16 wirelessly transmits a report signal to datacenter 12 so that the AI algorithm can ascertain the driver's emotional state based on the report signal and on the algorithm's prior training with previous sensor data.

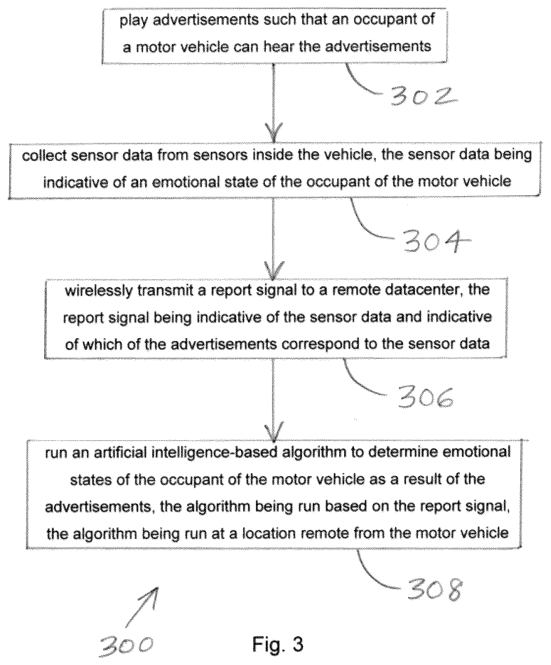

FIG. 3 illustrates one embodiment of a method 300 of the invention for evaluating advertisements in a motor vehicle. In a first step 302, the advertisements are played such that an occupant of the motor vehicle can hear the advertisements. For example, advertising content may be played on a radio or online streaming service of infotainment system 30 that an occupant of the motor vehicle 14 can hear.

In a next step 304, sensor data is collected from sensors inside the vehicle. The sensor data is indicative of an emotional state of the occupant of the motor vehicle. For example, the driver may be observed and monitored with cameras 18 or other sensors such as microphones 20, occupancy sensors 22, biometric sensors 24, keypad touchscreen 26 and vehicle operation sensors 28. The captured images and utterances of the driver may indicate whether the driver is happy (e.g., the driver smiling) or annoyed (e.g., the driver frowning).

Next, in step 306, a report signal is wirelessly transmitted to a remote datacenter. The report signal is indicative of the sensor data and indicative of which of the advertisements correspond to the sensor data. For example, the report may be wirelessly transmitted via the Internet from processor 16 to datacenter 12, and may include all sensor readings obtained during an identified advertisement and within the threshold time period after the advertisement has stopped playing.

In a final step 308, an artificial intelligence-based algorithm is run in order to determine emotional states of the occupant of the motor vehicle as a result of the advertisements. The algorithm is run based on the report signal at a location remote from the motor vehicle. For example, based on the historic data of AI models, the current sensor data can be interpreted by an AI algorithm at a remote datacenter 12 to ascertain what emotions the driver is feeling.

While this invention has been described as having an exemplary design, the present invention may be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

What is claimed is:

1. A motor vehicle, comprising:
a plurality of sensors configured to:
collect data indicative of emotional states of an occupant of the motor vehicle; and
transmit sensor signals including the data;
an infotainment system configured to play advertisements such that the occupant can hear the advertisements; and
an electronic processor communicatively coupled to the sensing devices and to the infotainment system and configured to:
receive the sensor signals; and
wirelessly transmit a report signal indicative of the received sensor signals and indicative of which of the advertisements correspond to the received sensor signals, the report signal being wirelessly transmitted to a remote datacenter configured to run an artificial intelligence-based algorithm to determine the emotional states of the occupant of the motor vehicle as a result of the advertisements, the algorithm being run based on the received sensor signals.

2. The vehicle of claim 1 wherein the electronic processor is configured to keep a record of which of the sensor signals are received during which of the advertisements.

3. The vehicle of claim 1 wherein the sensor signals and the advertisements each include a time stamp, and the electronic processor is configured to use the time stamps to match each of the sensor signals to a corresponding one of the advertisements.

4. The vehicle of claim 1 wherein the report signal is indicative of which of the advertisements correspond timewise to the received sensor signals.

5. The vehicle of claim 1 wherein the artificial intelligence-based algorithm is trained by use of machine learning.

6. The vehicle of claim 1 wherein the sensors include a camera and a microphone.

7. The vehicle of claim 1 wherein the sensors include biometric sensors.

8. A method of evaluating advertisements in a motor vehicle, the method comprising:
playing the advertisements such that an occupant of the motor vehicle can hear the advertisements;
collecting sensor data from sensors inside the vehicle, the sensor data being indicative of an emotional state of the occupant of the motor vehicle;
wirelessly transmitting a report signal to a remote datacenter, the report signal being indicative of the sensor data and indicative of which of the advertisements correspond to the sensor data; and
running an artificial intelligence-based algorithm to determine emotional states of the occupant of the motor vehicle as a result of the advertisements, the algorithm being run based on the report signal, the algorithm being run at a location remote from the motor vehicle.

9. The method of claim 8 comprising the further step of keeping a record of which of the sensor data are collected during which of the advertisements.

10. The method of claim 8 wherein the sensor data and the advertisements each include a time stamp, the method comprising the further step of using the time stamps to match the sensor data to a corresponding one of the advertisements.

11. The method of claim 8 wherein the report signal is indicative of which of the advertisements correspond timewise to the sensor data.

12. The method of claim 8 comprising the further step of training the artificial intelligence-based algorithm by use of machine learning.

13. The method of claim 8 wherein the sensors include a camera and a microphone.

14. The method of claim 8 wherein the sensors include biometric sensors.

15. An advertisement evaluation arrangement for an automotive environment, the arrangement comprising:
a motor vehicle including:
a plurality of sensors configured to:
collect data indicative of emotional states of an occupant of the motor vehicle; and
transmit sensor signals including the data;
an infotainment system configured to play advertisements such that the occupant can hear the advertisements; and
an electronic processor communicatively coupled to the sensing devices and to the infotainment system and configured to:
receive the sensor signals; and
wirelessly transmit a report signal indicative of the received sensor signals and indicative of which of the advertisements correspond to the received sensor signals; and
a datacenter disposed remote from the motor vehicle and configured to:
receive the report signal; and
run an artificial intelligence-based algorithm to determine the emotional states of the occupant of the motor vehicle as a result of the advertisements, the algorithm being run based on the received report signal.

16. The arrangement of claim 15 wherein the electronic processor is configured to keep a record of which of the sensor signals are received during which of the advertisements.

17. The arrangement of claim 15 wherein the sensor signals and the advertisements each include a time stamp, and the electronic processor is configured to use the time stamps to match each of the sensor signals to a corresponding one of the advertisements.

18. The arrangement of claim 15 wherein the report signal is indicative of which of the advertisements correspond timewise to the received sensor signals.

19. The arrangement of claim 15 wherein the artificial intelligence-based algorithm is trained by use of machine learning.

20. The arrangement of claim 15 wherein the sensors include a camera and a microphone.

21. The arrangement of claim 15 wherein the sensors include biometric sensors.
